(19) United States
(12) Patent Application Publication
(10) Pub. No.: US 2019/0336118 A1
(43) Pub. Date: Nov. 7, 2019

(54) INSTRUMENTS AND SURGICAL TECHNIQUES FOR DISC PREPARATION

(71) Applicants: Javier GARCIA-BENGOCHEA, Jacksonville, FL (US); Marc VON AMSBERG, Waxhaw, NC (US)

(72) Inventors: Javier GARCIA-BENGOCHEA, Jacksonville, FL (US); Marc VON AMSBERG, Waxhaw, NC (US)

(21) Appl. No.: 16/403,949

(22) Filed: May 6, 2019

Related U.S. Application Data
(60) Provisional application No. 62/667,302, filed on May 4, 2018.

(51) Int. Cl. A61B 17/02; A61B 17/16; A61F 2/46

(52) U.S. Cl. CPC A61B 17/025; A61B 17/1671; A61B 2090/062; A61B 2017/0256; A61F 2/4611

(57) ABSTRACT

A positive stop distractor, and arrays thereof, each instrument adapted with a generally flat/planar paddle and one or more mechanical stops on the planar paddle. And a modular distractor used to provide sustained distraction of a joint space, particularly a spinal disc space, including a distractor handle, a body, and a retrievable shim, and an optional cable for retention and retrieval of the shim.

### RELATED APPLICATIONS AND PRIORITY CLAIM

This application claims the benefit under 35 U.S.C. § 119(e) to U.S. Provisional Application No. 62/667,302 filed May 4, 2018, the entirety of which is incorporated herein by reference.

### FIELD

The present application describes various exemplary devices, systems and surgical techniques for achieving access to a site within the body, particularly the spine. More particularly, the present application describes instruments for accessing a surgical site in the body, particularly the spine. In some exemplary embodiments, the instruments are useful for accessing the spine for one or more purposes of penetration of the annulus of an intervertebral disc, distraction of a disc space to provide enhanced access to the space, stabilization of a distracted disc space, and removal of disc material, particularly in the lumbar spine. According to such embodiments, the present invention overcomes shortcomings in the art.

### DESCRIPTION OF THE RELATED ART

As with most other areas of surgery, is it preferable when operating on the spine to employ the least invasive surgical approach possible for achieving correction to minimize trauma and associated pain and blood loss experienced by the patient, to improve recovery time and outcomes, and to reduce operating room time and costs. And it is desirable to complete the surgery as time efficiently as possible. In all modes of approach, one or more special retractors and tubes are typically used to dissect and displace tissue and expose the vertebrae, and other instruments are used to release the annulus and open the disc space, remove disc material, and prepare the space to receive an implant. Thereafter, one or more implants may be inserted in the prepared space, typically together with one of a variety of bone graft and osteogenic materials. According to various modes of spinal disc access, surgeons use a variety of instruments, including among them distractors that have a generally paddle like shape with long handles for manipulation, and in some instances, mechanized rotation, of the paddle within the disc space. These distractors are typically used in conjunction with other instruments for penetrating the portion of the annulus that is proximal to the surgeon, and yet other instruments for achieving and maintaining distraction of the disc space during one or more of the removal of disc material and introduction of a fusion or replacement disc device, or other tissue manipulations and implantations. The use of a multiplicity of devices can increase the time and overall complexity of a surgery and can also introduce additional risks associated with insufficient disc removal, suboptimal placement of an implant, and a potentially life-threatening risk of penetration of portions of the annulus that are across from the site of entry. In this latter aspect, there is a risk that the disc annulus could be penetrated adjacent to a nerve or vasculature, leading to nerve damage and possible internal bleeding and even death.

There is a need for surgical instrumentation and techniques that provide multiple functionalities, particularly during procedures for accessing intervertebral disc space. Such instrumentation would provide one or more of disc access, disc removal and distraction of adjacent vertebra and would include features to minimize the risk of distant disruption of the annulus.

### SUMMARY

In one embodiment according to the disclosure, positive stop distractors include an elongate body with a length and width dimension, a proximal end having a handle, and at a distal end having a distal paddle that includes one or more mechanical stop features.

In another embodiment according to the disclosure, modular distractors used to provide sustained distraction of a joint space, particularly a spinal disc space, include a distractor handle, a body, and a retrievable shim. In some embodiments, the modular distractor also includes a cable for retention and retrieval of the shim.

Embodiments of the present invention are not in any way limited in use for any particular mode of spinal access, and thus, may be used in any mode of access, including, but not limited to, a posterior-lateral approach for spinal surgery, anterior (ALIF), posterior (PLIF), transverse (TLIF), lateral, and extreme lateral (XLIF). Embodiments of the present invention should also not be limited to the spine and may be used in other orientations and other surgical sites within the body.

### BRIEF DESCRIPTION OF THE DRAWINGS

Features and advantages of the general inventive concepts will become apparent from the following description made with reference to the accompanying drawings, including drawings represented herein in the attached set of figures, of which the following is a brief description:

FIG. 1 shows a front view of an array of positive stop distractors in accordance with the disclosure, the array including six graduated positive stop distractors, each having the same length dimension, and each having a proximal handle engagement feature and a distal paddle that is characterized by a flared distal tip and an array of stop seat apertures;

FIG. 2 shows a front view of a single positive stop distractor selected from the array shown in FIG. 1;

FIG. 3 shows a side view of the single positive stop distractor shown in FIG. 2;

FIG. 4 shows a bottom view (from the flared distal tip) of the single positive stop distractor shown in FIG. 2;

FIG. 5 shows a top view (from the proximal engagement feature) of the single positive stop distractor shown in FIG. 2;

FIG. 6 shows a perspective view of the single positive stop distractor shown in FIG. 2;

FIG. 7 shows another perspective view of the single positive stop distractor shown in FIG. 2;

FIG. 8 shows another perspective view of the single positive stop distractor shown in FIG. 2;

FIG. 9 shows a front view of another single positive stop distractor selected from the array shown in FIG. 1, the instrument including a mechanical stop engaged in one of the arrays of stop seat apertures;

FIG. 10 shows a side view of the single positive stop distractor shown in FIG. 9;

FIG. 11 shows a perspective view of the single positive stop distractor shown in FIG. 9;

FIG. 12 shows a side view of the mechanical stop shown in FIG. 9;

FIG. 13 shows a front view of the mechanical stop shown in FIG. 9;

FIG. 14 shows a top view of the mechanical stop shown in FIG. 9;

FIG. 15 shows a perspective view of the mechanical stop shown in FIG. 9;

FIG. 16 shows a perspective view of an embodiment of a modular distractor of FIG. 1;

FIG. 17 shows an exploded perspective view of the modular distractor of FIG. 1;

FIG. 18 shows a top plan view of the modular distractor of FIG. 1;

FIG. 19 shows a bottom plan view of the modular distractor of FIG. 1;

FIG. 20 shows front, side, back and side plan views of the modular distractor of FIG. 1; and

FIG. 21 shows exploded front, side, back and side plan views of the modular distractor of FIG. 1.

### KEY TO REFERENCE NUMERALS

10 array of positive stop distractors

100 positive stop distractor

110 handle portion

120 elongate body

125 paddle neck

130 stop seat aperture

140 paddle

150 lateral edge

160 distal tip edge

165 distal tip edge corner

170 mechanical stop

180 cylindrical top

182 grasping flats

190 threaded portion

200 modular distractor

210 distractor handle

220 body

222 body width dimension

224 body depth dimension

225 male extension

226 cable conduit

230 retrievable shim

232 shim width dimension

234 shim depth dimension

235 female receiver

236 shim cable conduit

240 shim engagement seat

250 cable

260 cable capture

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION

The general inventive concepts will now be described with occasional reference to the exemplary embodiments of the invention. The general inventive concepts may be embodied in different forms and should not be construed as limited to the embodiments set forth herein. Rather, these embodiments are provided so that this disclosure will be thorough and complete, and will fully convey the scope of the general inventive concepts to those skilled in the art. Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts.

As described herein above, there is a need for devices and systems that overcome the shortcomings in the art pertaining to surgical access, particularly access for spinal surgery. In view of this need, the embodiments of instruments and techniques provided herein address a variety of objects and advantages. The present application describes various exemplary instruments, and surgical techniques for achieving surgical access to a site within the body, particularly the spine. In some exemplary embodiments, the system and device components are useful for accessing the spine for one or more purposes of neural decompression, manipulation, removal, and replacement and reinforcement of intervertebral discs, particularly in the lumbar spine.

Positive Stop Distractor

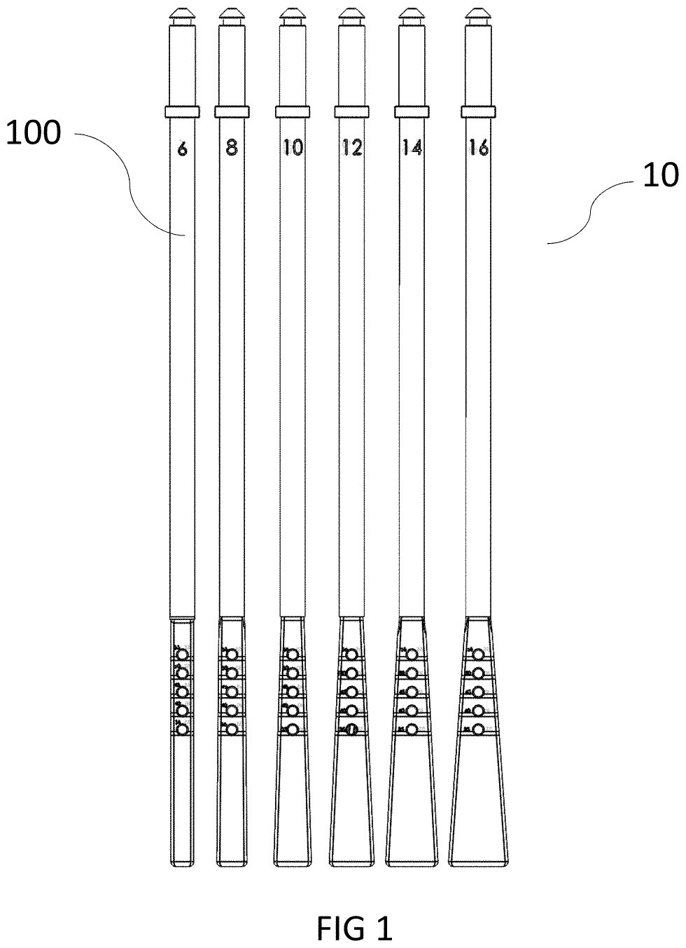

Referring now to FIG. 1, in accordance with the various embodiments, provided are positive stop distractors, each positive stop distractor 100 having an elongate body with a length dimension, and including at a proximal end having a handle engagement feature with a releasable handle, and a distal end with a distal paddle 140 that includes one or more mechanical stop features. FIG. 1 shows an array 10 of positive stop distractors 100, the depicted array 10 varying in dimensions for accommodating a range of varied width spinal discs. Each positive stop distractor 100 is adapted with a paddle 140 at the distal end of an elongate body 120 and includes at its proximal end a handle portion 110, the positive stop distractor 100 having a central axis that is generally rectilinear along the elongate body 120 and at least a portion of the paddle 140. The paddle 140 is insertable in a first orientation in a disc space with its opposing planar faces facing the adjacent vertebral end plates and is rotatable around the central axis of the elongate body 120 so that the paddle 140 faces are essentially or approximately perpendicular with the adjacent vertebral body endplates. In the depicted embodiment, the paddle 140 is generally flat with opposing planar faces.

The paddle 140 includes at least one mechanical stop feature 170 at a stop seat aperture 130 located on the paddle neck 125, the mechanical stop feature 170 extending away from one or the other of the faces of the paddle 140 so as to interfere with the edge of one or the other of the adjacent vertebral bodies and thereby to prevent movement of the paddle 140 into the disc space once the mechanical stop feature 170 has contacted the edge of a vertebral body adjacent to the disc space. The paddle 140 may have a narrow width or may be flared, such that the extent of the flare of a paddle 140 will influence the extent of distraction of the adjacent vertebrae upon rotation of the instrument within the disc space. The extent of the flare of the paddle 140 and its angle relative to the proximal end of the paddle 140 will provide the extent of lordosis of the distracted disc space.

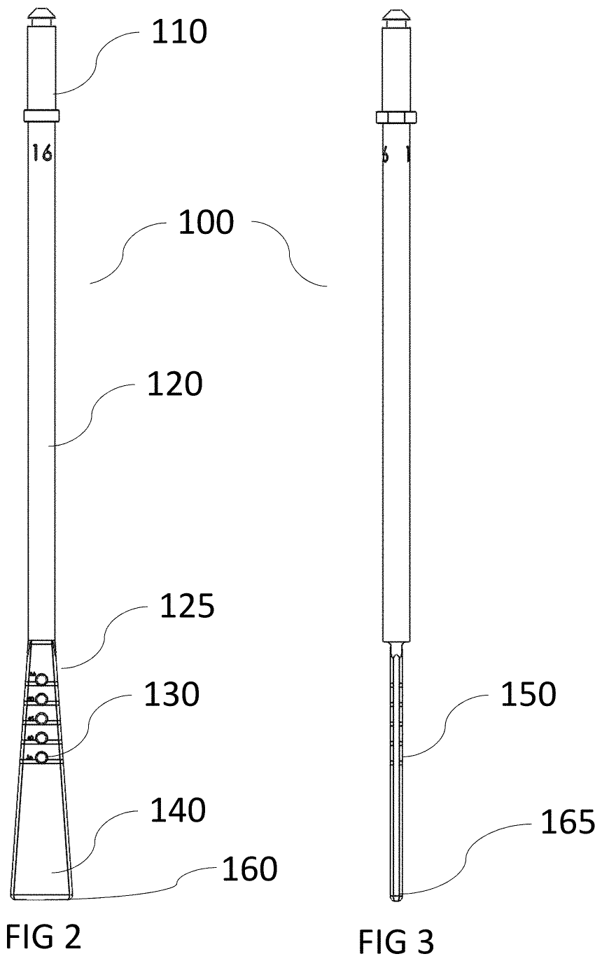

Referring again to the drawings, as shown in FIG. 2, for example, the elongate body 120 has a cross sectional shape that is selected from circular, square, and triangular, and an elongate body 120 width dimension that is greatest at the paddle 140's distal tip edge 160. The embodiment depicted in FIG. 2 has a circular cross-sectional shape. The distal paddle 140 extends from a proximal paddle neck 125 to a distal tip edge 160. The proximal paddle neck 125 is adjacent to the elongate body 120. In the depicted embodiment, the paddle 140 is generally planar and has dimensions including paddle 140 length, paddle 140 thickness and paddle 140 width. In some embodiments, the paddle 140 is flared (has a reverse tapered shape), such that the paddle 140 flares from the proximal paddle neck 125 to the distal tip edge 160. In some embodiments, such as those shown in the drawings, the flare is continuous. Of course, it will be appreciated that in other embodiments, the flare may be discontinuous, and in yet other embodiments, the width of a paddle 140 may be the same from the paddle neck 125 to the distal tip edge 160, while the width dimension may be greater than the width dimension of the elongate body 120. It will be appreciated that in some embodiments, the paddle 140 may be other than flat and may include one or more features or textures on one or other of the opposing faces and may have a shape, for example a wedge shape that is oriented along the long axis, on one or the other of the opposing faces.

Referring now to FIG. 3, the paddle 140 has a lateral edge 150 that runs from the distal tip edge 160 to the proximal paddle neck 125, the distal tip edge 160 having a thickness dimension that is selected from the same as the body width dimension of the elongate body 120, greater than the width dimension of the elongate body 120, or less than the body width dimension of the elongate body 120. In the depicted embodiment, the thickness dimension of the paddle 140, as shown at the lateral edge 150, is less than the width dimension of the elongate body 120. Referring again to FIG. 3, the lateral edge 150 is one of flat (squared), radiused, beveled, and chamfered. As shown in the represented embodiment, the lateral edge 150 is generally flat with slightly radiused edges. This aspect enables rotation within the disc space without cutting into or scraping the endplate surfaces while including a flat surface that allows stable retention of position of the paddle 140 between the vertebral endplates to maintain distraction.

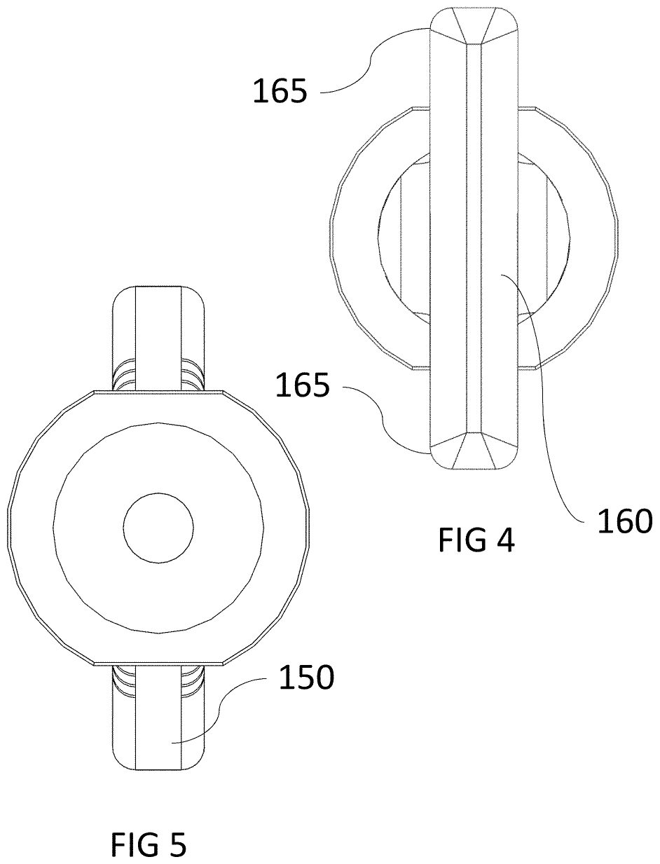

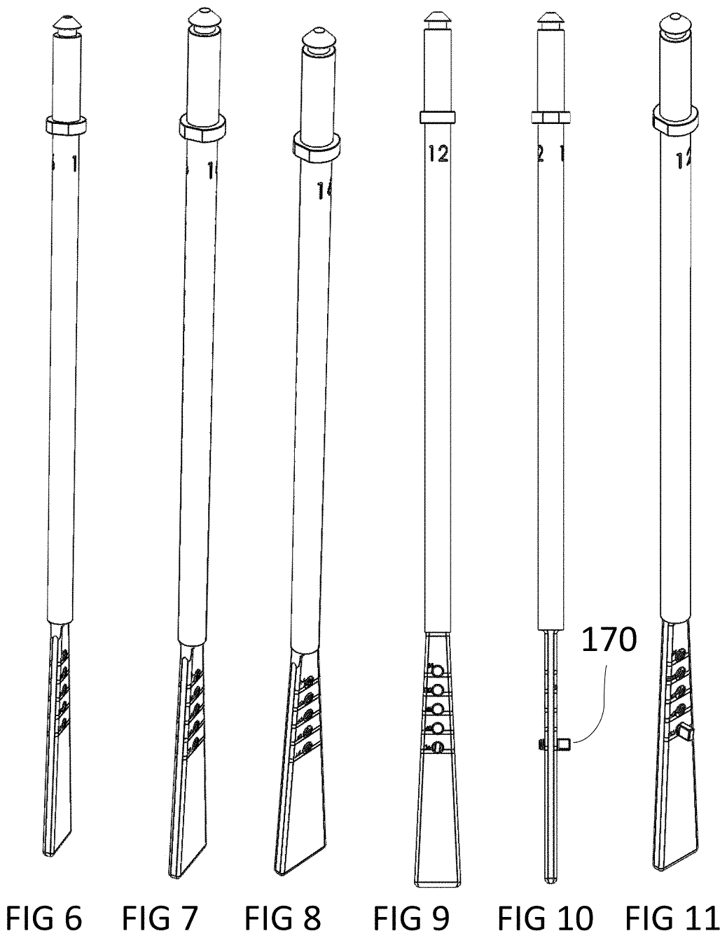

Referring now to FIG. 4, in the various embodiments, the distal tip edge 160 is one of flat (squared), radiused, beveled, and chamfered. Thus, in some embodiments, the distal tip edge 160 is blunt, and in other embodiments, the distal tip edge 160 is a cutting edge. In use, the cutting edge will facilitate penetration of the near annulus and also removal of disc material.

Referring now to FIG. 9 and FIG. 10, each paddle 140 is adapted with at least one mechanical stop 170 for contacting the edge of a vertebral body so as to block advancement of the paddle 140 into the disc space beyond the position of the mechanical stop 170. It will be appreciated that embodiments lacking such a stop, as with some of the distractor instruments known in the art, can be advanced to any depth within a disc space, even to a depth that would penetrate the contralateral annulus (at any surface on the disc annulus that may be from 0 degrees to about 90 degrees from the axis defined by the initial point of entry of the instrument into the disc space). In some embodiments, such as the instrument shown in the drawings, the mechanical stop 170 is removable, and engageable within one of an array of stop seat apertures 130 on the paddle 140, such as in the depicted embodiments on the paddle neck 125. In other embodiments, a mechanical stop 170 may be mechanically fixed or unitary with the instrument. In other embodiments, there may be only one stop seat aperture 130, or more or fewer stop seat apertures 130 than shown in the depicted embodiments.

In use, the mechanical stop 170 ensures fixed positioning of the paddle 140 at a desired depth in the disc space. This fixed positioning allows for enhanced distraction of the disc space for receiving an intended implant or other material, particularly where the distraction is intended to match the lordosis feature of an implant to the anatomically correct or desired degree of lordosis of the treated disc space. The fixed positioning further benefit includes protection of the contralateral and peripheral annulus from inadvertent penetration by the lateral edge 150 or distal tip edge 160 of the paddle 140.

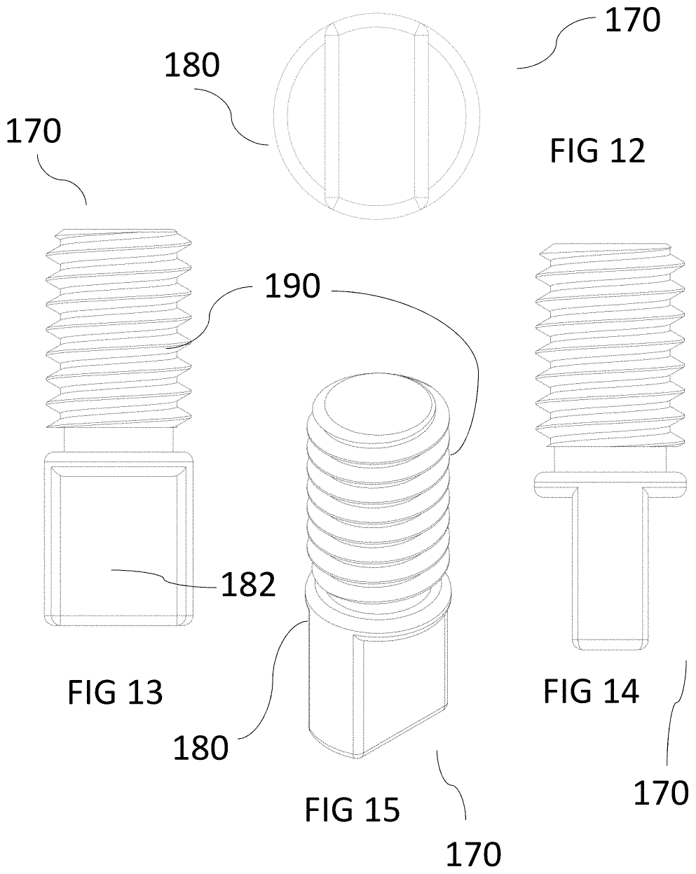

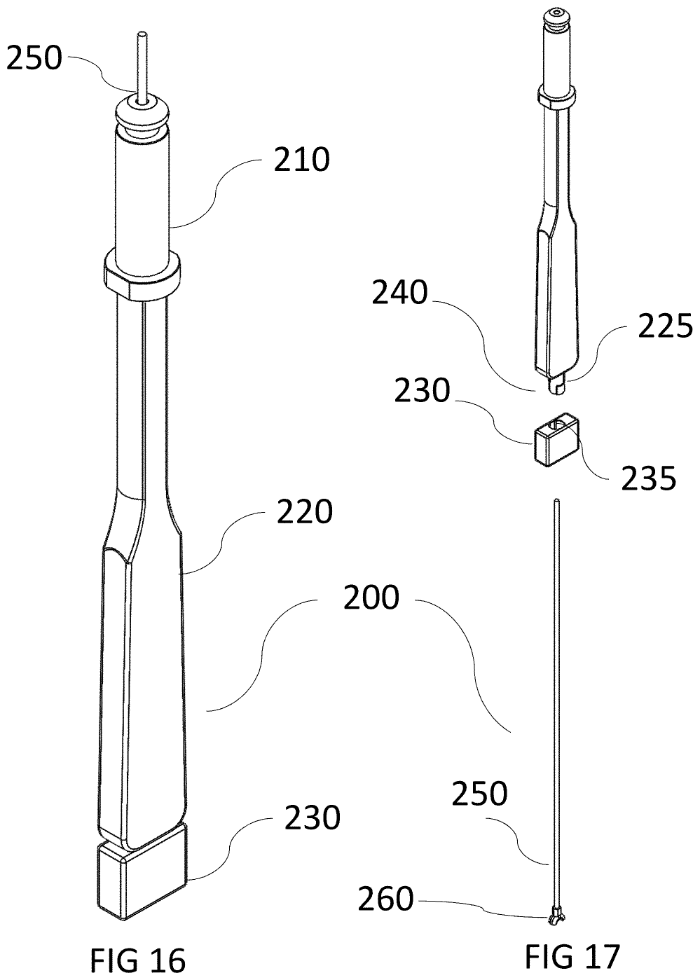

Referring again to FIG. 9 and FIG. 10, the mechanical stop 170 is engaged within a stop seat aperture 130 in the paddle 140 and extends away from at least one face of the paddle 140. FIG. 12-FIG 16 show various views of the mechanical stop 170 that is shown in FIG. 9 and FIG. 10. The mechanical stop 170, as depicted, has a threaded portion 190 for threaded engagement in a female threaded feature of a stop seat aperture 130 in the paddle 140. The mechanical stop 170 also includes a cylindrical top 180 with opposing grasping flats 182 that allow for griping with fingers or a tool to affix and remove the stop from a stop seat aperture 130. It will be appreciated that the overall shape of the mechanical stop 170 is not intended to be limited by the depicted embodiment, and as such, the stop may have any of a variety of shapes. Likewise, it will be appreciated that the fixation engagement feature of the mechanical stop 170 may be any one of fixation and fasteners known in the art and thus may include Morse taper female and female engagement features, snap fit, and other engagement means that are not limited to threaded engagement.

Referring again to FIG. 1, the positive stop distractors 100 may be provided in an array, such as the depicted array, which includes six graduated positive stop distractors 100. As shown, the paddle 140 widths vary in a graduated manner from 6 mm to 26 mm in 2 mm increments. Thus, the depicted array in FIG. 1 includes paddles 140 that have a distal width at their distal tips of 6, 8, 10, 12, 14 and 16 mm. In the depicted embodiment, the narrowest width has a 0° taper (i.e., its width is the same as the width of the elongate body). As the width increases across the array, the taper increases, up to a maximum 8° included angle. This angulation provides lordosis during distraction that would roughly correspond with the degree of lordosis of a selected implant. It will be appreciated that the degree of angulation could vary differently. And in some alternate embodiments, the overall width of a paddle 140 may not vary from the proximal paddle neck 125 to the distal tip edge 160 and thus a paddle 140 could have a fixed width without any angulation from proximal to distal (not shown).

As shown in FIG. 1, the representative array shows a set of six instruments all of the same length and all with the same paddle 140 length, wherein the instruments vary in terms of the angulation of the flared heads and the width of the distal end. It will be understood that an array could have more or fewer instruments, and any or a combination of the various instrument dimensions as described herein may vary across the array.

In accordance with some embodiments, as depicted in the drawings, the paddle 140s have a distal width at their distal tip edges of, in some examples, from 6 to 16 mm. Thus, in various embodiments, the paddle 140s have a distal width at their distal tip edges and may be as little as 3 mm and as much as 22 mm, including 3, 4, 5, 6, 7, 8, 9, 10, 11, 12, 13, 14, 15, 16, 17, 18, 19, 20, 21, 22, 23, 24, 25, and 26 mm, including increments and intervals therein and therebetween.

In accordance with the various embodiments, as depicted in the drawings, the paddle 140s have a paddle 140 length of about 80 mm from the distal end. Thus, in various embodiments, the length of the paddle 140 from the proximal paddle neck 125 to the distal tip edge 160 may be as little as 15 mm and as much as 120 mm, including 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, and 120 mm, including increments and intervals therein and therebetween.

In accordance with the various embodiments, as depicted in the drawings, the paddle 140s have a reverse taper (flare) along the paddle 140 length of the taper from about an angle of about 0° up to about 15° from the center axis of the instrument, and in some embodiments up to about 8°. Thus, in various embodiments, the taper angle increases from about 0 to 1, 2, 3, 4, 5, 6, 7, 8, 9, 10, 11, 12, 13, 14, and 15° from the center axis of the instrument, including increments and intervals therein and therebetween.

In accordance with some embodiments, as depicted in the drawings, the paddle 140s have one or more of a radiused lateral edge 150 and a radiused distal tip edge 160. Thus, in various embodiments, a contour of the paddle 140 distal tip edge 160 may have a radius from about 0.1 mm to about 1.5 mm, including 0.1, 0.2, 0.25, 0.5, 1, and 1.5 mm, including increments and intervals therein and therebetween.

In accordance with some embodiments, as depicted in the drawings, the paddle 140 has a distal tip edge 160 that is beveled or pointed suitable for cutting, particularly for penetrating the annulus.

In accordance with some embodiments, as depicted in the drawings, the corners of the paddle tips (corners of the flared tips) include a 2 mm radius. Thus, in various embodiments, a contour of the distal tip edge corners 165 of a paddle 140 distal tip edge 160 may have a radius in cm and increments in between including 0.2, 0.25, 0.5, 1, 2, and 3 mm, including increments and intervals therein and therebetween.

In accordance with some embodiments, as depicted in the drawings, the width (diameter) of each elongate body 120 is 6.3 mm. Thus, in various embodiments, the width of the elongate body 120 may be as little as 3 mm and as much as 3 cm, including 3, 4, 5, 6, 7, 8, 9, 10, 11, 12, 13, 14, 15, 16, 17, 18, 19, 20, 21, 22, 23, 24, 25, 26, 27, 28, 29, and 30 mm, including increments and intervals therein and therebetween.

In accordance with some embodiments, as depicted in the drawings, the overall length of the instrument from the proximal handle portion 110 to the distal tip edge 160 is 21.5 cm. Thus, in various embodiments, the length of the instrument from proximal to distal may be as little as 15 cm and as much as 35 cm, including 15, 16, 17, 18, 19, 20, 21, 22, 23, 24, 25, 26, 27, 28, 29, 30, 31, 32, 33, 34, and 35 cm, including increments and intervals therein and therebetween.

In accordance with some embodiments, as depicted in the drawings, each paddle 140 has an array of five stop seat apertures 130, every 5 mm, starting at 45 mm from the distal tip edge 160. It will be appreciated that there may be more, or fewer stop seat apertures 130, and the spacing may be the same, or different. Likewise, the stop seat apertures 130 may have different shapes and may be adapted to receive any of a variety of different stops, the feature being most advantageous in its function to block progress of the instrument into the disc space.

Modular Distractor with Retrievable Shim

Referring again to the drawings, FIG. 16, shows a modular distractor 200 that includes a distractor handle 210, a body 220, and a retrievable shim 230. In some embodiments, the modular distractor 200 also includes a cable 250 for retention and retrieval of the shim 230. In general, the modular distractor 200 and retrievable shim 230 have overall dimensions that range as further described herein to accommodate a range of varied width anatomical spaces, such as intravertebral spinal disc spaces.

The embodiment shown in the drawings represents a modular distractor 200 having a body 220 that tapers from distal to proximal, having a maximum body width dimension 222 and maximum body depth dimension 224 at the distal end and tapers adjacent the distractor handle 210. In cross section, the distal end of the body 220 has a generally rectangular cross-sectional shape, and at the proximal end near the distractor handle 210, the body 220 has approximately a square cross-sectional shape. The embodiment shown in the drawings represents a modular distractor 200 that has a body 220 that tapers from distal to proximal, having a maximum body width dimension 222 and maximum body depth dimension 224 at the distal end and tapers adjacent the handle 210. In cross section, the distal end of the body 220 has a generally rectangular cross-sectional shape, and at the proximal end near the handle 210, the body 220 has approximately a square cross-sectional shape.

Referring again to FIG. 16, the embodiment of the retrievable shim 230 shown in the drawings has a body 220 that is defined by width and depth dimensions 232, 234 to define a cross sectional shape that is generally rectangular and closely matches the shape of the body 220 at its distal end. In some embodiments, the width and depth dimensions 232, 234 of the shim 230 are the same as the body 220 width and depth dimensions 222, 224 at the distal end of the body 220. In some particular embodiments, as further described herein the shim 230 dimensions are slightly larger than those of the body 220 in at least one of the shim width 232 or the shim depth 234 dimension, and in some such embodiments, the shim width dimension 232 is greater than the maximum body width dimension 222. Further, in some particular embodiments, the shim 230 has corners and edges that are squared and with or without a bevel.

As depicted, the peripheral edges of the body 220 and the shim 230 are beveled, and at least the distal edge of the body 220 has curved edges while the edges of the shim 230 are generally squared. It will be appreciated that the edges may be other than beveled, and may be squared, squared with a bevel, or curved, or curved with a bevel, or chamfered, or the edges may comprise combinations of these.

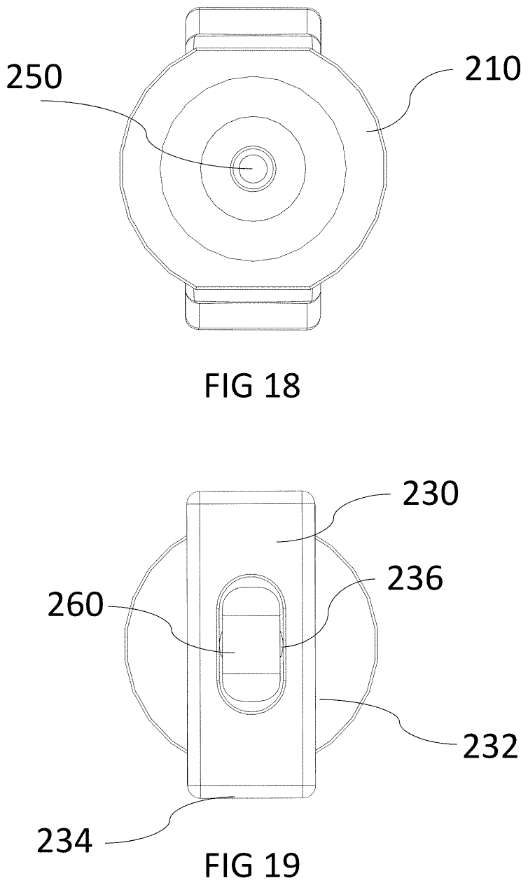

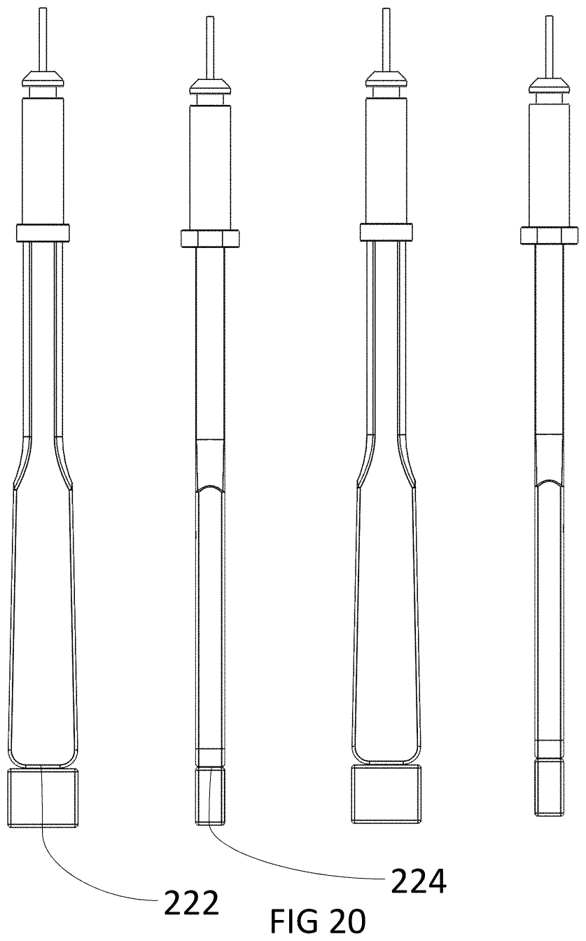

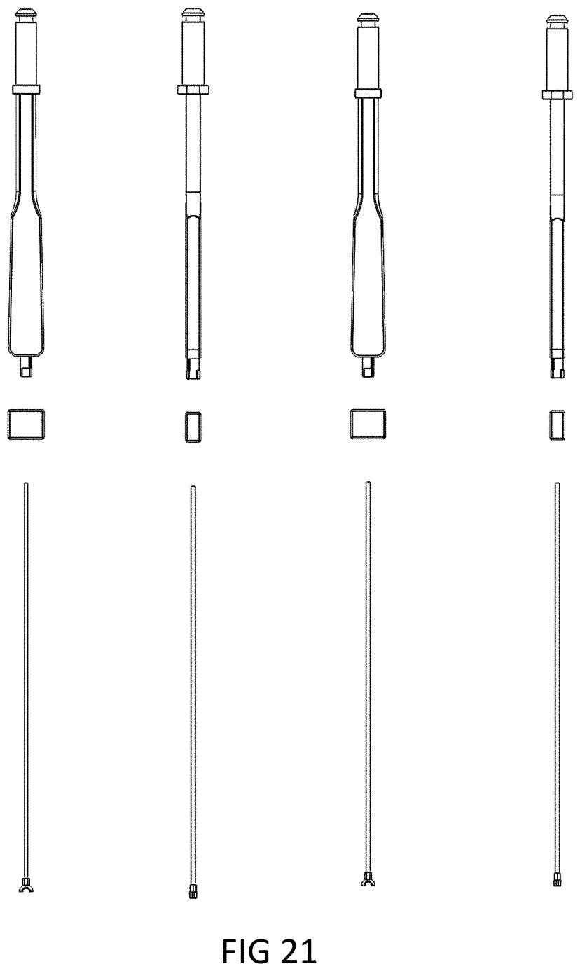

In the depicted embodiment, each of the modular distractor 200 and retrievable shim 230 have faces that are substantially smooth and planar. It will be appreciated that surface features, textures and surface shapes other than planar are possible and the depicted embodiment is not in any manner limiting with respect to these features. FIG. 20 and FIG. 21 show each of the sides of a representative modular distractor 200, and FIGS. 18 and 19 show the respective proximal (top) and distal (bottom) ends of the representative embodiment.

Referring now to FIG. 17, assembly of the modular distractor 200 is achieved by engagement between at least the shim 230 and the body 220 via a shim engagement seat 240. In the depicted embodiment, the engagement is achieved with a male extension 225 from the body 220 that forms the shim engagement seat 240 that is received in a female receiver 235 on the shim 230. In use, the male extension 225 and the female receiver 235 are engageable by insertion of the male extension 225 into the female receiver 235 in an unlocked state when the width dimensions of each of the body 220 and the shim 230 are other than parallel with each other. Once engaged in an unlocked state, upon rotation of one of the body 220 and the shim 230, the engagement seat 240 is fixed by locking between the male extension 225 and the female receiver 235 in which state the faces of the body 220 and shim 230 are aligned in parallel. It will be appreciated that in other embodiments, the fastening means may be other than as shown, and may include one or a combination of snap fit, interference fit, cam locking and other mechanisms for releasably locking components as is generally known in the art. Further, the features may be flipped in their orientation such that the male feature is on the shim 230 and the female feature is on the body 220. Advantageously, placement of the male extension 225 on the body 220 ensures that the shim 230 has minimal features that may interfere with access to the disc space during a surgical procedure. Engagement between each of the body 220 and the shim 230 may be by rotation in either a clockwise or counter clockwise motion, and disengagement is achieved by the opposite rotation. In some particular embodiments, the convention of clockwise rotation for engagement is used and counterclockwise for disengagement. In some embodiments, it will be appreciated that there may be greater ease in rotation and removal of the body 220 if its dimensions are smaller, at least in the body width dimension 222 than the at least body width dimension 232 of the shim. Accordingly, in some embodiments, the shim 230 is sized as slightly larger than the body 220.

In some embodiments, the modular distractor 200 includes a cable 250 that is useful for retaining the shim 230 and body 220 loosely together prior to use of the device. The cable 250 is also useful for securing the body 220 and the shim 230 together during insertion and removal of the modular distractor 200 from the disc space. And, the cable 250 can be beneficially used to facilitate retrieval of the shim 230 from the disc space, in some instances without the need to reengage the shim 230 with the body 220. As such, the cable 250 may be grasped by an operator and simply pulled to withdraw the shim 230 after its use for distraction has been satisfied. Referring again to the drawings, as shown in particular in FIGS. 17 and 19, the cable 250 includes a cable capture 260 feature which is a mechanical structure that may or may not be removable from the cable 250 and serves to secure the cable 250 to the shim 230. According to the depicted embodiment, the female receiver 235 of the shim 230 includes a through bore that forms a shim cable conduit 236 through which the cable 250 is passed with the cable capture 260 configured to interfere with the distal end of the shim cable conduit 236 to preclude passage of the cable capture 260 through the shim cable conduit 236, thereby securing the cable 250 to the shim 230. In other embodiments, another form or shaped cable capture 260 may be used and it may be affixed with the shim 230 by another feature or means.

In the embodiments comprising a cable 250, the cable 250 is passed through the body 220 and exits at the proximal end through or adjacent the distractor handle 210. The cable 250 may be controlled at that location by grasping such that when the shim 230 is initially deployed by disengagement from the body 220, it may still be controlled via grasping the cable 250 proximal end. In some embodiments, the distractor handle 210 may include another actuator that facilitates control of the cable 250.

As described herein above in reference to the positive stop distractors 100, various dimensions of distractors are described in terms of width, length dimensions and these are generally applicable to the modular distractors 200 as described above. One of ordinary skill in the art will realize that the specific dimensions are determined based on what is generally understood and practiced in the art and that the inventive features particularly with respect to the modular distractor are not in any manner limited by dimensions.

Retractor Handles

According to some embodiments, the various distractors also comprise one or more of an adjustable and releasable stabilization handle for positioning and stabilizing the positive stop distractor 100, the handle being adjustable and releasable to avoid interference with other surgical instruments, and may include a vertical shift component for dorsal to ventral vertical adjustment and positional locking to avoid interference between components of the insertion assembly and with other surgical instruments. In accordance with some embodiments, the handle 110 is releasably engageable with or may be fixed to the proximal end of the instrument. And in some embodiments, the instrument includes at its proximal end a snap fit engagement feature (male) that is insertable into a female receiver 235 of a handle 110 to releasably fix engagement. The examples are, of course, non-limiting, and the relative length and shape of the handle 110 may vary. Thus, the handle 110 may be any one or more of removable, rotatable, pitched to the left or the right relative to the body 220 of the retractor, and combinations of these. In the various embodiments, the handle 110 is adapted for holding the instrument during insertion into the disc space, penetration of the annulus, and rotation of the paddle 140 to distract the disc space.

Representative Surgical Technique

A representative embodiment of a surgical technique includes the following steps, the order of which is not intended to be limiting:

Position patient (for example, for posterior access, prone and generally parallel to the floor) on a suitable surgical table.

Obtain a radiographic scan to measure ventral to dorsal height of target vertebral space. Calculate total distance from the intended access portion of the disc space (for example, for a posterior access, the anterior to posterior or ventral to dorsal height), to select the desired depth limit of the instrument and thus select the stop seat aperture 130 for inserting the mechanical stop 170 into a stop seat aperture 130 of the paddle 140.

Select instrument from the array to provide the desired paddle 140 length and taper (distal flare). Insert instrument and advance to contact annulus. Freehand or using a mallet, engage the distal tip edge 160 to penetrate the near annulus to gain access to the disc space. Advance the paddle 140 distally until the stop is engaged at the edge of one of the vertebra bordering the disc space. Rotate the paddle 140 to 90 degrees from its planar aligned orientation with the vertebral endplates to provide for distraction of the disc space. Confirm progress radiographically.

When using a modular distractor 200 with retrievable shim 230, the technique is as described above except that upon rotation in a clockwise motion relative to the operator of the modular distractor 200 within the disc space, the body 220 may thereafter be rotated in a counter clockwise manner, with or without use of a proximal lock actuator, to release the shim 230 from its engagement with the body 220 so that it is retained in the disc space to maintain distraction. The body 220 is removed from the space and the cable 250 is withdrawn from the cable conduit 226 of the body 220. During the surgical procedure, the cable 250 is displaced to the side. The shim 230 can later be retrieved from the space by reengagement of the cable 250 within the cable conduit 226, passage of the body 220 into the disc space, rotation of the body 220 into reengagement via the shim engagement seat 240 with the shim 230, and extraction of the reassembled modular distractor 200 from the disc space. Alternately, the shim 230 may be extracted from the disc space simply by grasping and pulling the cable 250, or by grasping the shim 230 with forceps or another surgical instrument, without need to reengage the body 220 with the shim 230. And it will be appreciated that in those embodiments where a cable 250 is not employed, either the body 220 or another surgical instrument may be used to remove the shim 230.

As used herein, the singular forms “a,” “an,” and “the” are intended to include the plural forms as well, unless the context clearly indicates otherwise. The term “proximal” as used in connection with any object refers to the portion of the object that is closest to the operator of the object (or some other stated reference point), and the term “distal” refers to the portion of the object that is farthest from the operator of the object (or some other stated reference point). The term “operator” means and refers to any professional or paraprofessional who delivers clinical care to a medical patient, particularly in connection with the delivery of care.

With respect to any references herein that may be made relative to a human patient, the terms “cephalad,” “cranial” and “superior” indicate a direction toward the head, and the terms “caudad” and “inferior” indicate a direction toward the feet. Likewise, the terms “dorsal” and “posterior” indicate a direction toward the back, and the terms “ventral” and “anterior” indicate a direction toward the front. And the term “lateral” indicates a direction toward a side of the patient, the term “medial” indicates a direction toward the mid line of the patient, and away from the side, the term “ipsalateral” indicates a direction toward a side that is proximal to the operator or the object being referenced, and the term “contralateral” indicates a direction toward a side that is distal to the operator or the object being referenced. More generally, all terms providing spatial references to anatomical features shall have meaning that is customary in the art.

Unless otherwise indicated, all numbers expressing quantities, properties, and so forth as used in the specification, drawings and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

References to visualization using radiography as described in the exemplary techniques herein are merely representative of the options for the operator to visualize the surgical field and the patient in one of many available modalities. It will be understood by one of ordinary skill in the art that alternate devices and alternate modalities of visualization may be employed depending on the availability in the operating room, the preferences of the operator and other factors relating to exposure limits. While confirmation of instrument placement in the course of the technique is appropriate, the frequency and timing relative to the sequence of steps in the technique may be varied and the description herein is not intended to be limiting. Accordingly, more or fewer images, from more or fewer perspectives, may be collected.

One of ordinary skill will appreciate that references to positions in the body are merely representative for a particular surgical approach, and according to the exemplary embodiments herein, are suitable for any number of animal patients, including humans and other species. Of course, the type of surgery, target tissue, and species of patient may be different than is disclosed in the exemplary embodiments described herein. Further, all references herein are made in the context of the representative images shown in the drawings. Fewer or additional generic instruments may be used according to the preference of the operator. Moreover, references herein to specific instruments are not intended to be limiting in terms of the options for use of other instruments where generic options are available, or according to the preference of the operator.

Thus, while the disclosed embodiments have been described and depicted in the drawings in the context of the human spine, it should be understood by one of ordinary skill that all or various aspects of the embodiments hereof may be used in connection with other species and within any species on other parts of the body where deep access within the tissue is desirable.

Further, while various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts and features into additional embodiments and uses within the scope of the general inventive concepts, even if such embodiments are not expressly disclosed herein. Additionally, even though some features, concepts and aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

I claim:

1. A modular surgical distractor, comprising:
an elongate body having opposing generally planar faces along a length dimension between proximal and distal ends, a handle portion at the proximal end, and at the distal end a maximum width dimension, and a maximum depth dimension, and a center elongate axis, along the body, the distal end having a generally rectangular cross sectional shape
a shim that is engageable with the elongate body by releasable engagement at the body's distal end, the shim having opposing generally planar faces along a length dimension, and having each of a width dimension and a depth dimension
each of the elongate body and the shim comprising engagement features that form an engagement seat for releasable engagement between the body and the shim between locked and unlocked configurations, the locked configuration actuated by rotation of one of the body and shim from an antiparallel arrangement of the body and shim width dimensions to a parallel arrangement of the body and shim width dimensions.

2. The modular surgical distractor according to claim 1, wherein the modular distractor in the locked configuration comprises the body and shim engaged and oriented with their opposing faces aligned as parallel, and in the unlocked configuration comprises the body and shim engaged and oriented with their opposing faces aligned generally antiparallel, and wherein the actuation of locking between the body and the shim comprises rotation in a first direction of one of the body and the shim around the central axis of the elongate body so that the planar faces are essentially perpendicular, and wherein the actuation of unlocking between the body and the shim comprises rotation of one of the body and the shim in a second direction, the shim is disengagable from the body.

3. The modular surgical distractor according to claim 2, wherein the actuation of locking between the body and the shim comprises rotation of the body in a counterclockwise direction, and wherein the actuation of unlocking between the body and the shim comprises rotation of the body in a clockwise direction.

4. The modular surgical distractor according to claim 1, wherein one or more of the width and depth dimensions of the shim is the same as or greater than the width and depth dimensions of the elongate body.

5. The modular surgical distractor according to claim 1, wherein the body tapers from distal to proximal.

6. The modular surgical distractor according to claim 1, comprising a removable handle.

7. The modular surgical distractor according to claim 1, wherein the shim has at a distal end corners and edges that are radiused or beveled.

8. The modular surgical distractor according to claim 1, wherein the body includes at a bottom surface of its distal end a male extension and the shim includes at a top surface at a proximal end a female receiver, the male extension and female receiver engageable by insertion of the male extension into the female receiver in an unlocked state when the width dimensions of each of the body and the shim are other than parallel with each other, each of the male extension and the female receive including engagement features that lock engagement when the body and shim are aligned in parallel.

9. The modular surgical distractor according to claim 1, wherein the shim is sized as slightly larger than the body.

10. The modular surgical distractor according to claim 1, further comprising a cable that is passable through a conduit in at least one of the body and shim and along the axis of the body.

11. The modular surgical distractor according to claim 10, wherein the cable is fixedly engaged with the shim.

12. The modular surgical distractor according to claim 1, wherein the body has at its proximal end an engagement feature for releasable engagement with the handle portion.

13. The modular surgical distractor according to claim 1, wherein, in use in an intervertebral disc space, the modular distractor is insertable in a first orientation with the body and shim opposing faces aligned in parallel and facing adjacent end plates within the disc space, and the assembled body and shim are rotatable in a first direction around the central axis of the elongate body so that the planar faces are essentially perpendicular with the adjacent vertebral body endplates, and upon rotation of the body in a second direction, the shim is disengagable from the body allowing removal of the body from the disc space and retention of the shim therein.

14. A positive stop distractor comprising:
an elongate body having a length dimension and a width dimension, and a center elongate axis, the elongate body comprising
a proximal end having a handle, and
a distal end having a generally flat and planar distal paddle that includes one or more mechanical stop features positioned along the paddle above the distal end
wherein, in use in an intervertebral disc space, the instrument is insertable in a first orientation with its opposing planar faces facing adjacent end plates, and is rotatable around the central axis of the elongate body so that the planar faces are essentially perpendicular with the adjacent vertebral body endplates, and
wherein the one or more mechanical stop features is adapted to extend away from one or the other of the planar faces of the paddle so as to interfere with the edge of one or the other of the adjacent vertebral bodies so as to prevent movement of the paddle into the disc space once the mechanical stop has contacted the edge of a vertebral body adjacent to the disc space.

15. The positive stop distractor according to claim 1 wherein the paddle is flared, having a taper angle from the center axis that originates at the proximal neck, the taper angle from 0 to 10 degrees.

16. The positive stop distractor according to claim 1 wherein the elongate body has at its proximal end an engagement feature for releasable engagement with the handle.
